SEQUENCE LISTING (7 sequences)

SEQ ID NO: 1 - DNA, 15941 bp, Homo sapiens
cgactctacaacaacaacaaaaatacaaaaattagcccctgtaggcccagttacttgggaggttgaggtgggaggatcgcttgagcctgggaggttgaggctgcagtgagctatgagctgcaccactgtactccagcctgggtgacagggcaagactctgtctcaaaaaacaaaaacaacaacaaaaactttattcctaaaaagtgctgacccagagataagaagcaagcacatggttggtaaaatgacatcagtgagcttgctttatgcagggttgccccaaaccttcaatttgtaaaactcaaaatatctggaaagcttggtgaggcgaggcgggactgtaactcagcactcaccgcagggtgagacccttagggcagcgggtgctacactgaacaaagcggtgaacagccccctcgtgagagggggttcttggcagatgacgccagcagctgacaaacaggcaaggcaggccgggcacggtggctcacgcttgtaatcccagcactttgggagtccaaggtaagcggatcacaaggtcaagagatcgagaccatcctggtctacatggtgaaaccccatctctactaaaaaatacaaaaattagctgggcgtggtgatgcacacttgtagtcccagctactcagggggctgaggcaggagaatcacttgaaccgggaaggccgaggttgcagtgagccgagattgtgctgctgcactccagcctggcgacagagcgagactctatctcaaaaaaaaaaaaaaaaaaaaaaaacccagcaaggcagggacgccagcagggcacgtgctgaggagacaggcgcgagcaccaaaggtgcagggcggcctcttagccggagtccaggaggcctttgaggggagagggcttctggcaaaccccaggggtggggatgagcagtgacaggggccctgggggctgggatgtgacagggcagcgtggcagtgtctggtggcccctcccgcaggccatcaccatcgaggctgagccaagagctgatggcagccgccggaccacccgctatgacatcgacatgaccaagtgcatctactgcggcttctgccaggaggcctgtcccgtggatgccatcgtcgaggcacgtgaggcccccgggtgggagggggcctgaggctcatcagcagggcctaaccaccgtccctgcacctcaacatgcagggccccaactttgagttctccacggagacccatgaggagctgctgtacaccaaggagaagttgctcaacaacggggacaagtgggaggccgagatcgccgccaacatccaggctgactacttgtatcggtgacgccccaccggcccgcagcccctgctgcccaataaaaccactccgaccccacggcctcttgtcttgactctggtggtccagggtggggccgttcccggcctgcctgaggcctgtggtgcctccaactggactaggtcgccacctagtctttatgctctctggagtcctgctgccccagccctgccttcctgtagactccagggccccaccaggctctcatggctagaccacaggccgcagcatccctgtcaccctgcctcttcctgggcgtcttccccaaggcccagcgatgccagaggtcaccacagacctgccgttcccactctcccgggtgtcctggctggggcctgggcctcctgtctcccaagagccatccacttgtaagacgcttaggacttggagcctcccaagacctcaggctgaagaacagcaagtggcagaaactcaggccggggcatccttgctgggggtaggaggaagcctgcttggggtgacagtcacacgagggtgacacaggccctgctggccctgagctggcctgcgtccagccacatcctctttcctgtgttccctacgccagggtgctggcccggccactgctcaccacttcctgctttgctggcccagcgacacggtggggtttccctagggccacacacaagccccttgcccatataatggccaaaagcagcccccctgaacttgtgctaagtactgtggaaggatttcacttcatttgcagtgaggaaaccagctccgagaggtgaaagggcttgctcagggccatgcagggcacatcccataataaaactcagttcagagaccttgaccccccccatctaccatcatttccaaaagtgctttttacaaagcactactttgtaaaaagctacttcttcgaaagaacaagtttcctggcccagtgagtgttggaaatgctccagctgctgtgtgcctgctggcaggtactgcatggtagacattcaaggctctgagaaggcctgcagcaaaaacaccagcctctcccagcccattcccagggagcttttctcaggtctaacacctgggcctccttcctagaccaggcattgccatctaatgccaaagtggccaggctcccgcttgggccccacagcctgtggcactggggtgagaagtaactgcaggaggcgaggggaagcctggagcccgggaagtgtccacaggtctggcccgcagctcatcttggccactgatgaaaacaggtgttctttccaaaaacaaggcaggcgcaggactcctgaagcaccgccggtttagcttttcccagacgtaaccgccgtcagagcgtggcatggagcctgtagctgtgcactgaatacttctattccaagcgggtcagtctttccagctcaccgctgggttctttcccttaccgtggtctcccaggcacttactgctgagaccccactcaggctgtctggttgtatcaggccccgaccctgtcagaacaggcctatggcgcccaaccacaagtcccccaattttctgaggaaccacagctgagcttggctgtgactcagtttccaggcccggctctggggctccacttgtccacccctgccgcagccccacaggcctgtgctcgctgtgtggggcttagggatcaccagagctgaagggcccctggcctccagtgggaatgccccaggcctgtccaaagtgcaggcagctggacggtcgtgagtcccagcttccaggcactaggacctgatgacgcccatggtgctgacctggagcaaggtcacagtgacacccccctctaccccccgcggctgcagctgcaggtggagctgacagctggcctctggccccagggtatctcccagggcctgctgggtgcggggcgctgggcagaacaaggcagcatggcctggccctgtgtcctgctgccagtgaagcaggcccagagtggggcagccacccacccaagggccagagcggggcctctccagggtggtcaagagcaggaaggtgtaagcaccggcgggtttcactgccgcccaagggaggggtcccggaggtgggggagcatcctggggaccccaagcccatgagcctgcgaatgttggggtcccatcagccagaggcgcacaagcctcgaagtgagaggagagcagggtctcatctcctatcctgagctgggcctgagcggagctgcccctgcatcggggccagcatagacgcgcgcggttggcagtggtctgttatccgctcctttctccaggcctcctctccgggattaagcgcctcaggacccgtccgccaggccacagcctgggctcaacctctcccagacttcctggactccagtggagccttgggccgggggcggggcgtccctggccctccccgtcgtcccgcctgcccggaaaggagtgagcggcgcttagtcccgggctggcgggagtgcagttctgagtcccgcccggcgtgcgcggagcgggcagccagcagcggaggcgcggcgcgcagcacacccggggtgagtgcccctgccggggtctgcggagccccagaatttcctgcccaccctcttccccctgccccggggaggagatcctggcgccccacccagttggctccgtgaatgcagctcagcccgccaccccgcagggtgtcaggccttgcggggggccacggaggatccccaagggaggaggagaaggatggggtgtttaccaacacagcccagcgttcccgggcctggtgtgcgcggagctttccccgctgctgcttccttggagggagggacccattctacagatggggaagctgagaccacacaactaggaagtgtcagagctggagaccagctgggtcatttgactccagagcccaccccatttggaatggagggggcgccagaccctctcctactgccttcccatcatctggaggggagggcagaggtgcagagagacctgctgggttcctggaggggccaggaaaggggcacagggtgaggagtgggctggagcctggaccccagggccgcaggatggaggcgtattcatttacaccataaaagtggctgggtgacccctggctagagcggggaccaagggggacaggggtagggggtgagccagtggagcctcacatgccaggcacgggggctgatcacgtccccacacctttccccccgcccattacaggttgttcccccaccccctgggaagtgctggaaggagcccggccctcgagccctcggcatagtggctgtgggagccctgatggccctgagccaggggtgacccctccccagatccccagagctgattccccacacctccttatttggccagcacagccccaaagggagccaggtcagacacaaccctcctctgagctcaccctttgtgctgggcacataacattggactttacctctccaagtgtcagtggagtcctgtatgaaacaaaagcaccgtaccccctccccaagggtttctccccccagggattagtgtgaggtcgtgtggatgaagggattcagttgaggctctggttatttttgtcacctccctagatcctgtctctgtcaggggtgccacgtcccattcacccaaagaggaagtagaggcacagagagggtgagagacttgcccaaggtcacacacccaatctcagcctgggctccaagtccctctgccctgtggccgggcctggtccttagggatggaacccagccccgctgccccctgccctgcccacctgcagccttgggccatctgctcagtgttccctcgttccctccttctctcctttcccaggctggggctggggctgcgtttccctgtttagagttaagtaaaccagacccaggggaggaagtgactggctgggcaggcggggccctgaccaggcctgtcccctccccctaattttcgagctaagcaactgattcccacagaggggccctgggccctgagagcagtggctttggagctggaggggccccagcatccagggcgggttggaggtggcctggcacctgcccccaccccacatcggccttgagcaaggccccgggcccttccccgccattctgtagtttcacttttgttggtttcaacacctggaaggctgcggctgctgccgggcggcccgggcctcaggggacagcccccccacagcagtgctccctggctagccccagggtacccggtggagatgcccgctcctaccactcagcacatgcagctccacagggagcagcgagggctccagccacccccggggcagccctgggcctaagtgcctgggccagtggggtcctgtctagggtgacggcagcccgagaggagggaggccagagtgaaggtgggggtggagccggtgtcactgggagacgctggaggtaggggcgggggtccccatgggcagagcaagccaggagagagcttgaggtcggcctgagggtgggcttcggggtctcccagtcccactgcttactttctgcgtgacctcagcaccgcatcaagcctgcccaggcctcagtttccacctctgtcagatgggggtggtaacagcagcttccaggattcagtgagtgaaggtgcacaggtgcccgtggttgggaactgtctgtggtctgcccctgactggcccccatccgtgtccacccacaggaccatgggctccatgttccggagcgaggaggtggccctggtccagctctttctgcccacagcggctgcctacacctgcgtgagtcggctgggcgagctgggcctcgtggagttcagagacgtgagttgggtgggcaggcgtgggaagggggctactgccaaggttagcccggaggccggtccaggatggggactgccccccctccgccatagggccctggccccatttgaaccccagcggcccctgccatgggcactgctcatgggaagcccgcagctgaggcccctgagctggctcctccccataccctcctggggcatggggtctggtctgtgctctgatctgcgtcttgtggctcccagggcactccacacctttctggaggaggcagctaaggcctggggagagtcaggcctgggctctagggtgaggagctccctgacccccttcccgggacactcacccctccgtgtggcacccacagctcaacgcctcggtgagcgccttccagagacgctttgtggttgatgttcggcgctgtgaggagctggagaagaccttcagtgagttggtcccaggcctacattccaggcaggcttcctggaggaggcatgggccaagtttgatctgaaaggaagagtctgggtttgccaggtggaaggcagagaagggaggtatatgcagggccctgcagggccaagacagagcagctgggatctgtgaagtgtgcttgcgaaggtggctgccttgtgtgggctgagcagagggcctagggcagggctggctggggaggcctcgaatacagcataagagcccaggacttcgtcctgtgggcgccaggaagccacaggggtttctaagcagggaagaggcacagacagctttctcctctacagctgggctggggtgggtggggcccaggatctagggtagggtacccctaaggagcaggcaaaagggacccctgccctgggcaacccctctcaggaggagcctgtgggggcaccaaggcaggggcatggggcaggcagggttttgtgtttgctggtgcagtttacagcttttcaatgatcaggcagacacaggactcacgccagctccggtcccaagccctgcagcgcaagggccggcctgtggggagacctcaggccctcttctgagtggtggccaagggactggggagggagagtgagcccagacctgagctcagggaggggagcacagcttctggatgaaagtttggccgggattttctggccacctccacctggtgaatccagcagctggtggccgatggagtttggggcagcaggtggggccctcaactgttgagacaacctcaactgcaccccactcccgttcctctgcgcccagccttcctgcaggaggaggtgcggcgggctgggctggtcctgcccccgccaaaggggaggctgccggcacccccaccccgggacctgctgcgcatccaggaggagacggagcgcctggcccaggagctgcgggatgtgcggggcaaccagcaggccctgcgggcccagctgcaccagctgcagctccacgccgccgtgctacgccagggccatgaacctcaggtcagctcccacccaggcaggagactggggggctggggaggggctgtccagcctagggctcatggtgcttctgggttcctagctggcagccgcccacacagatggggcctcagagaggacgcccctgctccaggcccccggggggccgcaccaggacctgagggtcaagtgagtgagggatgacctcatgccctttctggccagcccagaacccctggccagtcgctgggctgggccaggctgagctccgactccttgtccagtgctctccccaggctggccccgcctcctccttcaggcccggaacttcccacagtcccaagccctagccctagggggttctcctcttctggtcctgcccgggaggcctcctgccttcccctgtgggcagggccagtgtgcccaattgcccgattgcccgtgctgggcagggtcctgcccggggggcctggtgggggaggcagggcaggaggttggagcagccctgcccagccccgtggccgccagctttgtggcaggtgccgtggagccccacaaggcccctgccctagagcgcctgctctggagggcctgccgcggcttcctcattgccagcttcagggagctggagcagccgctggagcaccccgtgacggtgagcagctggcgctgggctggggggtcctgggcagagcgggaccccagagtcagctgagcctgctctgcagggcgagccagccacgtggatgaccttcctcatctcctactggggtgagcagatcggacagaagatccgcaagatcacggactggtgagtcactgggaacacccgccccaccgccctgctccccaggcccctgctgtcacctcccagcccgccctatcgtgactcctccccatgagctcccttcagactcagagtctcgtagctgtgtccttctggcccctcacgcagcgcatcctccctccagcttccactgccacgtcttcccgtttctgcagcaggaggaggcccgcctcggggccctgcagcagctgcaacagcagagccaggagctgcaggaggtgggtgcccccggccttccggaggcgggtgtaggaggtgggtgcccccggcctcccggaggtgggtgcaggaggtgggtgccctggcctggcctccaggaggtgggtgcaggaggtgggtgccctggcctggcctccaggaggtgggtgcaggaggtgggtgcccccggcccagccaccccacctgcctgcccagcccccgctgactgcccactctggcgcaggtcctcggggagacagagcggttcctgagccaggtgctaggccgggtgctgcagctgctgccgccagggcaggtgcaggtccacaagatgaaggccgtgtacctggccctgaaccagtgcagcgtgagcaccacgcacaagtgcctcattgccgaggcctggtgctctgtgcgagacctgcccgccctgcaggaggccctgcgggacagctcggtgagcagcctgaggcctcgccccctctccgcccgcccctcctaccaggccggggcgtttctgcctcacttccagcctctggctccctgcacctgggctttgcctaggggctccttctcctcccagcctcctccatggcccaccgcttccaggggagtctgccctgatggccctggcccagcaagccctaccctgactttcctaagtccctggaggctgcacaggatagttttaaagtcagaagcctcattccaatccaggcccccctgtgaccttaggcctgctcatggtctgtggaaccggtgtgatgcccctgcctccgggggtcacccgccgcgcacagcaggtgcttgcgggtgaggctgggctccttctcctgggccgtccctaggaggcagcatgcttgcctttcccaagcaattgccaatccatgtggtgtctttggggccccaccaagggcagagcagggctgatcatctcacgtcagagagaggggaaggggctgcccagtgagcccccacagggtctacatctccagctgggcctggctggagatcccagggtccctgaaggcccccgccaccgttctggtctgtctctgccctggcacccagatggaggagggagtgagtgccgtggctcaccgcatcccctgccgggacatgccccccacactcatccgcaccaaccgcttcacggccagcttccagggcatcgtggatgcctacggcgtgggccgctaccaggaggtcaaccccggtgagagccacggcatccttacccgtgtcctgggaggctcagctgccccactgggtgggtgtgagcctgagggggaggtatctccatcctgggcctggcctgccctcctgggatgaggggcacacatcccatcactctcaaggggctgttgggctctgcagccagtgtgaggctttatgtcggcccccagccaactcccccttgttgagtttacaagggagccggcacgggcttgtgcaaagaacagaggctgtgcggtgggcatggcccaggttggaagcccatctcgcctactttgagacatttcttagcttctctgagcctcagctgcctcacctgcaaaatggaaataatagcgcaaacctcagaggaatgttgggaagcttagtgactgttggagactgcaagctggagccaggggcagcctttggtgtggttgctttggttgcacagctttttttttttttttgagatagagtcttgctcagttgcccaggctggagtgcaatggcatgatctcggctcactgaaacctccgcctcccaggttcaagcgattctcctgcctcagcctcccaagtagctgggattacaggcacgcaccaccacgcctggctaatttcatatttttagtagagatggggtttcaccatgttggtcaggctggtcttgaacccccgacctcacgtgatccacccacacaggccgttgggggcccaagcaccctgactcttgcccactggatgaggcagggcccctccctgtcctgtgaccaagtcccctcccgacatgggctccaccacatcacacccatgctatgggccgggcccccactcagtgtcactgggggcactggggggtttgctgcttctgcagggccagtgtcctggggctggatctgggccccaacaggattcccctgcacgtgggcagaggctggtgcagctagagcatcacctggaggcccgtggcagatagaatgctgccagctcaaagcaccagccgtcggggcagcgtgtttgccagggaagctgtaacaaagtgccaccggctactggcttaaggggcagacacggtcccacagtcctggaggctggaagtccaagaccaaggtgtcagcaggggccgggggcactggctgatgcctgtgatctcagcactttgggaggctgtggtgggaggaggatctctggagtccaggagttcgagaccagcctgggcaacatagcaagcccttgtatcttaaaaaaataaaagatgatgaggtgttgtcagcaggagtggctccttctgaggctgtccaggaagatctgttcccggcctctctcctgggcttggagcggccgtcctcatcttcacacgctgttctccttctatgtgggtccaaatgttccttttcataaggacatcgggcacactggattggggcccactgtaatggccttatcttaacccagtgacctctgtaaggaccctgtctccaaagaaggtcacatctggagttcctgggggttgcgactccagtgtatcttttttggggtcacaacacagcccctgacagtgtgtatgttggggtcctcctgcctcccatgaggacttgttcccattttactgatgggaagtcgagacttcagagctgaagtgaccagctcggcctcccggaacttgccatgtgccagctggggctgcctgtggctccctgtcctctctggcttctgtgtggagctctgggaacagcaacagctgaagctgctaacttcctgtggctccagcagctggcccgtgctgcccgtggagaacttatgaagtggtctggtgttacctgcattttacagatggggaaatggaggctcagagaggtgaagtggctgggccaaggtcacacagctcctaagtggtgggttcttttttttttttttttttttcggagacagaatcttcgctctgtcgcgcaggctggagtgcagtggcgcgagctcggctcactgcaacctccgcctcccgggttcaaatgattctcctgcctcagcctcctgagtagctgggattacaggcacctgccaccatgcccagctaatttttctgtttttagtacaggcagggtttcaccgtgtgggccaggctggtctcgaactcctgacctcgggtgatgcccccccccccccgtctcggcctcccagagtgctgggattacaggcatgagccactgtgcctggctgctaagtgatgggttcttgactgcaggccagcagggcctccgtggcgtcttgggccgggaggcagatgctggtgtgttcgggggttcccagctcacccacctctgcccacagctccctacaccatcatcaccttccccttcctgtttgctgtgatgttcggggatgtgggccacgggctgctcatgttcctcttcgccctggccatggtccttgcggagaaccgaccggctgtgaaggccgcgcagaacgaggtgaggggcggggctggggtcctgatgagggtagcagggccaggcagcccctcaccacaccactgcccccccagatctggcagactttcttcaggggccgctacctgctcctgcttatgggcctgttctccatctacaccggcttcatctacaacgagtgcttcagtcgcgccaccagcatcttcccctcgggctggagtgtggccgccatggccaaccagtctggctggaggtgaggcccgggccccagcccggctgggggccccgcagcacccgcagccctgaccgccctcccctgcgttgccgcagtgatgcattcctggcccagcacacgatgcttaccctggatcccaacgtcaccggtgtcttcctgggaccctacccctttggcatcgatcctgtgagtcctgggatggagtgtccgtgggtggtgaaggcagctgggagtgggggctggggctcccctcggttcagccgtcctgcagcgctgtcgctgagcactcctgtgtgccaggccctttcctcagcacaagaggcagacaagcctcctgccctgcggagcctgcatcacagtcccctgagtgtgcggaggcaaagcgagaccagccaggaaccagtctcctgagtgtgcagaggcaaagcgagactggacaggaaccagcctgcagcttgcactgtgccaagcactgttcccaaccctctgtggaagtgattttcttttttcttttttttatttttggatatagagtctcattctgttgcccaggctggagtgcagtggtgtgatcttggctcactgcaacctccatctcccgggttcaagcgattcccctgcttcagcctcctgagtagctgggattacaggcgcccgccaccatgcctggctaatttctgtatttttagtagagacagggtttcaccatgttggctaggctggtctcgaactcctgacctcatgtgatccacctgcttcagcctctcgaagtgctgggattacaggcgtgagtcaccgcgcccagcctgattttctatttgaacctcgtaacaaccccgtgggcctcacattagtagtgtcctcacctaaagaagagactgaggcacagaggtcatgcccaaggtcacccaggctggcaccgtggcagctggcccatctgcgctctgttgcccctcggtgggtgggtgatggatgaggctgcaggctccgagggggaaaacagggtggtgagagagtgactcgggccggggacttcctggcagtgatggcgagggagcccctgagtccagcccacccctgctgccaccctagatttggagcctggctgccaaccacttgagcttcctcaactccttcaagatgaagatgtccgtcatcctgggcgtcgtgcacatggcctttggggtggtcctcggagtcttcaaccacgtgtgagggccaaggctgcccgggggacgggaggctggcaggccagagtgggccccagtgagcacacctccctcttgcccgccaggcactttggccagaggcaccggctgctgctggagacgctgccggagctcaccttcctgctgggactcttcggttacctcgtgttcctagtcatctacaagtggctgtgtgtctgggctgccagggccgcctcggcccccagcatcctcatccacttcatcaacatgttcctcttctcccacagccccagcaacaggctgctctacccccggcaggtgggctgcggctggtgggggccgggctcacacggcctcatggggaccccgcggtcacagggccactgggagctgcaagatcctcgtccgagaaacggggatgcaggccccgggccgtgcagacagggccgtcagaggtgatggtgtgcatctttagcaggtggcacaactggcactgggaaccgggggtcccttccctcaggagtccttatggaggcagaccctcacccagtgagggcacggagcccccgggggccctggagggaggagcgaccgcaggctctctgggccccgtgactgctgtgactcaggttcctttgcaggtgtgcacagcagggacgccctgactctcgccctctccctggcaggaggtggtccaggccacgctggtggtcctggccttggccatggtgcccatcctgctgcttggcacacccctgcacctgctgcaccgccaccgccgccgcctgcggaggaggcccgctgaccgacaggtgggaccggggcctaaggtgtggggggctgcttgcggagaggccactgtccggtgtgtccctgactcctcgcttcctgacagactcctgagtggccaggagcaggcctggcgggtggtgggggacctcctggggctggagtgctgccaacactgcctgctcatgccccaggaggaaaacaaggccgggttgctggacctgcctgacgcatctgtgaatggctggagctccgatgaggaaaaggcagggggcctggatgatgaagaggaggccgaggtgggtgcagtgccttcctgggggtgggacggctgaggccctgccggccctcactgcacccgccccgcagctcgtcccctccgaggtgctcatgcaccaggccatccacaccatcgagttctgcctgggctgcgtctccaacaccgcctcctacctgcgcctgtgggccctgagcctggcccacgcccgtgagtgacctggccaccgacggctggccccagctcctggcttctcacataccgctgctggctgggcgggttgcttctctctgggcctcagtttcccctctgtaaagtgggactgtccaaggaggtccctcgctggccccccgtgcagggagggcttcaggctgcggcaggtaggtagggggctggcaggcacccacttgccgttggcccccactgtctcctttgcttgcagagctgtccgaggttctgtgggccatggtgatgcgcataggcctgggcctgggccgggaggtgggcgtggcggctgtggtgctggtccccatctttgccgcctttgccgtgatgaccgtggctatcctgctggtgatggagggactctcagccttcctgcacgccctgcggctgcactggtgagcgaccacccactggcctgggctgctcaaggcgtgagttcccctcaccaacccctctgcttctcacccccagggtggaattccagaacaagttctactcaggcacgggctacaagctgagtcccttcaccttcgctgccacagatgactagggcccactgcaggtcctgccagacctccttcctgacctctgaggcaggagaggaataaagacggtccgccctggcagtgatgtctcgtctctcttccctcctttttagctgaggcaggtggcagggtagaaggcatggtgtaggatctgggacctcaaaagtcaaggcctggacctgagcagatctgcaataatgtcagactcttggggtgaaaaggggcagaaaagggccaaggagagatgtttccagcacattctaacttggagtgagcaaatgtcttcccaggccgggtcaggggagtgagctccccatccttggaggtgtgcacctggctgtgagcgggtgtctgggttactctctgttgggagtggtgccaatatccatgccctgg

SEQ ID NO: 2 - DNA, 21 bp, Artificial sequence
  Primer
acaaggcaggcgcaggactcc

SEQ ID NO: 3 - DNA, 21 bp, Artificial sequence
  Primer
cgggcctggaaactgagtcac

SEQ ID NO: 4 - DNA, 20 bp, Artificial sequence
  Primer
ttggggcagcaggtggggcc

SEQ ID NO: 5 - DNA, 23 bp, Artificial sequence
  Primer
agaggagaaccccctagggctag

SEQ ID NO: 6 - DNA, 19 bp, Artificial sequence
  Primer
gttcggggatgtgggccac

SEQ ID NO: 7 - DNA, 19 bp, Artificial sequence
  Primer
gcccataagcaggagcagg

(19) United States
(12) Patent Application Publication
(10) Pub. No.: US 2005/0176006 A1
(43) Pub. Date: Aug. 11, 2005

(54) Genetic markers for bone mass

(76) Inventors: Paolo Vezzoni, Milano (IT); Cristina Sobacchi, Milano (IT); Anna Villa, Milano (IT); Stuart Ralston, Aberdeenshire (GB)

Correspondence Address: NIXON & VANDERHYE, PC, 901 NORTH GLEBE ROAD, 11TH FLOOR, ARLINGTON, VA, 22203, US

(21) Appl. No.: 10/503,733

(22) PCT Filed: Feb. 4, 2003
(86) PCT No.: PCT/GB03/00470

(30) Foreign Application Priority Data
Feb. 6, 2002 (GB) 0202771.2

(51) Int. Cl. C12Q 1/68

(52) U.S. Cl. 435/6

(57) ABSTRACT

The invention provides an agent for preventing or treating arthritis, a cartilage protecting agent, a joint destruction inhibitor and a synovial membrane growth inhibitor comprising an anti-FGF-8 neutralizing antibody as an active ingredient, as well as a diagnostic agent of arthritis comprising an anti-FGF-8 antibody as an active ingredient and a method for judging arthritis using the antibody.

The present invention relates to methods for genetic analysis of bone mineral density and susceptibility to disorders which are related to bone mass. It further relates to materials for use in such methods.

### BACKGROUND ART

Genetic factors play an important role in the pathogenesis of osteoporosis—a common disease characterised by reduced bone mass, microarchitectural deterioration of bone tissue and increased susceptibility to fragility fractures ¹. Bone mineral density (BMD) is an important predictor of osteoporotic fracture risk and evidence from twin and family studies suggests that between 50%-85% of the variance in BMD is genetically determined ²⁻⁴. However the genes responsible for these effects are incompletely defined. BMD is a complex trait, which is likely to be regulated by an interaction between environmental factors such as diet and exercise several different genes, each with modest effects on BMD.

A wide variety of candidate genes have been studied so far in relation to BMD, including the vitamin D receptor ⁵, the estrogen receptor ⁶, and the COLIA1 gene ⁷. Current evidence suggests that allelic variation in these genes accounts for only a small portion of the variance in BMD however B indicating that most of the genes which regulate BMD remain to be discovered.

Linkage studies in humans have mapped three Mendelian traits that are associated with abnormalities of BMD to a region of chromosome 11q12-13. These are osteoporosis-pseudoglioma syndrome⁹; autosomal recessive osteopetrosis¹⁰ and high bone mass¹¹. This region of chromosome 11 was also found to be linked to BMD in normal female sibling pairs¹², indicating that allelic variation of genes within this region may play a role in regulating BMD.

Recent work has also shown evidence of linkage between a polymorphism at the TCIRG1 locus and femoral neck BMD in healthy premenopausal sib-pairs (Carn et al (2002) J Clin.Endocrinol.Metab 87:3819-3824).

However, the results from such linkage analysis are only able to localise the phenotypic effects to within regions of millions of base pairs and do not identify the gene or genes responsible for the phenotypic effect observed. Thus no clear association, as distinct from linkage, has previously been demonstrated between markers in this region and regulation of BMD in a normal population. The genotyping of such genetic markers would be useful as markers of bone mass and hence, for example, susceptibility to osteoporotic fractures.

### DISCLOSURE OF THE INVENTION

The present inventors have demonstrated that allelic variation in the TCIRG1 gene in 11q12-13 contributes to regulation of bone mass in normal individuals.

The TCIRG1 gene is known to encode a 116Kd subunit of the osteoclast specific vacuolar proton pump. It is a component of the vacuolar-ATPase complex expressed in the osteoclast ruffled border and is responsible for transport of H+ ions into the resorption lacuna, where the low pH plays a role in dissolving hydroxyapatite crystals¹⁷. TCIRG1 mutations have previously been shown to be present in approximately 60% of individuals with infantile osteopetrosis^{13;14}. However they were not know to be associated with regulation of bone mass in normal individuals.

Briefly, the present inventors studied the relationship between bone mineral density (BMD) and TCIRG1 polymorphisms in a population based cohort of several hundred perimenopausal Scottish women. They identified five novel polymorphisms at the TCIRG1 locus; two in the promoter; one in exon 4, one in intron 4 and one in intron 11.

The inventors demonstrated a significant association between the G9326A genotype and BMD at the lumbar spine (p=0.01) and femoral neck (p=0.03). G9326A is within the promoter, within a consensus recognition site for the AP1 transcription factor.

The association remained significant after correcting for age, weight, height, menopausal status/HRT use and smoking (p=0.008 for spine BMD and p=0.03 for hip BMD) and homozygotes for the “G” allele had BMD values significantly higher than individuals who carried the “A” allele at both spine (p=0.007) and hip (p=0.047). Subgroup analysis showed that the association between G9326A and BMD was restricted to premenopausal women, who comprised 50.6% of the study group.

The five polymorphisms showed strong and highly significant linkage disequilibrium with each other in the population, with the exception of C14242T where linkage disequilibrium was only observed with A14286G.

Thus it appears that common allelic variants (allele frequency >0.05) of the TCIRG1 gene can account for at least part of the heritable component of BMD, possibly by affecting peak bone mass. The TCIRG1 polymorphisms are thus useful as genetic markers e.g. for identifying people with low BMD, so that these individuals could be targeted for treatment to prevent osteoporosis.

### BRIEF DESCRIPTION OF THE INVENTION

At its most general, the present invention provides methods for assessing bone mass (e.g. peak bone mass) and particularly BMD (e.g. lumbar spine BMD or femoral neck BMD) in an individual, the methods comprising using a TCIRG1 marker, particularly a polymorphic marker to assess this trait.

In preferred embodiments these methods may be used to assess the susceptibility of the individual to disorders which are to some extent (wholly or partly) related BMD. Such disorders are hereinafter termed “BMD-related disorders” and the methods and materials herein may also be used for the diagnosis andor prognosis for them.

Preferably, the present invention is concerned with disorders associated with low BMD, especially osteoporosis and related disorders. For example, the methods of the present invention may be used to determine the risk of certain consequences of relatively low BMD, such as to determine the risk of osteoporotic fracture (McGuigan et al (2001) Osteoporosis International, 12, 91-96).

The method may comprise:
(i) providing a sample of nucleic acid, preferably genomic DNA, from an individual, and
(ii) establishing the presence or identity of one or more TCIRG1 (polymorphic) markers in the nucleic acid sample,
plus one or more further steps to calculate a risk of osteoporotic fracture in the individual based on the result of (ii).
   Predicting Risk of Osteoporotic Fractures

The methods of the present invention may be used to attribute a likely BMD value to the individual based on the result established at (ii).

Alternatively or additionally they may be used in prognostic tests to establish, or assist in establishing, a risk of (developing an) osteoporotic fracture, which is the major clinical expression of osteoporosis. Methods for making such predictions are well known to those skilled in the art and the present disclosure may be used in conjunction with existing methods in order to improve their predictive power. Other known predictors include BMD, weight, age, sex, clinical history, menopausal status, HRT use, various SNPs and so on. The diagnosis of osteoporosis (and prognosis of fracture) is reviewed by Kanis et al (1994) J Bone and Mineral Res 9,8: 1137-1141.

McGuigan et al (2001) supra disclose predictive methods based on a combination of bone densitometry and genotyping (in that case COLIA1 genotyping). Individuals were classified as either high or low risk on the basis of these two methods, which were inter-related but independently predicted risk of sustaining osteoporotic fractures. Thus, by analogy, the present TCIRG1 test may be predictive independently of BMD scores.

Marshall (1996) BMJ 312: 1254-1259 discloses a meta-analysis of how BMD measures predict osteoporotic fractures and attributed relative risk values and confidence intervals to various BMD measurements. The paper refers to a number of other risk factors for fracture. Cummings et al (1995) N Engl J Med 332: 767-73, also reviews risk factors (in that case for hip fracture in white woman)

All of these papers, inasmuch as they may be utilised by those skilled in the art in practising the present invention, are hereby incorporated by reference.

Thus preferred aspects of the invention will involve establishing or utilising one or more further measures which are predictive of osteoporotic fracture and defining a risk value (e.g. low, medium, high) or relative risk values or odds ratios (adjusted, for instance, against the population of that age and optionally sex) and optionally a confidence value or interval, based on the combination of these. Statistical methods for use in such predictions (e.g. Chi-square test, logistic regression analysis and so on) are well known to those skilled in the art. In a preferred embodiments a battery of tests (both genotyping and phenotyping) will be employed to maximise predictive power.

The methods may further include the step of providing advice to individuals characterised as being above low or medium risk, in order to reduce that risk (e.g. in terms of lifestyle, diet, and so on).

Particular methods of detecting polymorphisms in nucleic acid samples are described in more detail hereinafter.

Nucleic Acid Sample

The sample from the individual may be prepared from any convenient sample, for example from blood or skin tissue. The DNA sample analysed may be all or part of the sample being obtained. Methods of the present invention may therefore include obtaining a sample of nucleic acid obtained from an individual. Alternatively, the assessment of the TCIRG1 polymorphic marker may be performed or based on an historical DNA sample, or information already obtained therefrom e.g. by assessing the TCIRG1 polymorphic marker in DNA sequences which are stored on a databank.

Where the polymorphism is not intronic the assessment may be performed using mRNA (or cDNA), rather than genomic DNA.

Choice of Individual

Where the present invention relates to the analysis of nucleic acid of an individual, such an individual may be entirely symptomless, or may be one who has a BMD-related disorder, or is considered to be at risk from BMD-related disorder such as osteoporosis (e.g. by virtue of other determinants e.g. age, weight, menopausal status, HRT use etc. As described in the results below, although the association with preferred markers was demonstrated in the whole population, subgroup analysis revealed that the effect was primarily driven by an association in the premenopausal population. This would be consistent with a model whereby the TCIRG1 allele affects peak bone mass rather than postmenopausal bone loss,

The method may be used to assess risk within a population by screening individual members of that population.

Preferred Markers

It is preferred that the polymorphic marker is a single nucleotide polymorphism (SNP), which may be in an intron, exon or promoter sequence of the TCIRG1 gene. Preferably it will be a common allelic variant (allele frequency >0.05).

Preferred polymorphisms are as follows:
G9326A: situated in the promoter.
G9508A: situated in the promoter.
C14242T: situated in exon 4.
A14286G: situated within intron 4
G19031A: situated within intron 11.

It should be noted that all polymorphisms are, for convenience, numbered in relation to the latest sequence accession at the time of filing (LOCUS AP002807, 63433 bp DNA Linear PRI 24-JAN-2002, DEFINITION Homo sapiens genomic DNA, chromosome 11q clone:RP11-802E16, complete sequences—revised Jul. 5, 2002). Using an earlier accession (AF033033) 14242, 14286 & 190031 were at gene positions 3856, 3900 & 8645 respectively.

Annex I shows sequence of the TCIRG1 gene (as taken from a BAC clone). The promoter SNPs 9326 and 9508 are at positions 2648 and 2830 respectively. Based on the disclosure herein the skilled person is well able to identify the position of the polymorphisms of the invention in the TCIRG1 sequence.

Thus preferred SNPs for analysis are at any one or more of the following TCIRG1 gene positions: 9326, 9508, 14242, 14286, 19031.

More preferred are SNPs at position: 9326. The association between BMD and allelic variation at the G9326A site was highly significant at the spine (p=0.007) and at the femoral neck (p=0.03), after correcting for potential confounding factors including age, height, weight, menopausal status/HRT use and smoking.

Accordingly, in one embodiment the method of the present invention comprises assessing in a genomic DNA sample obtained from an individual one or more TCIRG1 SNPs selected from the SNP at position 9326, or a polymorphism in linkage disequilibrium with said SNP.

In a further embodiment the method may comprise assessing two, three, four or five of the TCIRG1 SNPs. Any suitable combination of one or more markers may be used to assess the BMD trait.

The method of the invention may comprise, in addition to assessing one or more TCIRG1 SNPs, or one or more polymorphisms in linkage disequilibrium with a TCIRG1 SNP, the assessment of other polymorphisms which are linked or associated with a BMD-related disorder.

Examples of such other polymorphisms include polymorphisms in the VDR gene and the COLIA1 gene (Uitterlinden, et al. (2001) Journal of Bone and Mineral Research).

Identity of Alleles

The assessment of an SNP will generally involve determining the identity of a nucleotide at the position of said single nucleotide polymorphism.

Preferred assessment of the SNP at position 9326 described above will establish whether or not the individual is homozygous for the G allele at these sites (and hence likely to have higher BMD).

For example, for SNP 9326, in relation to likely susceptibility to a disorder associated with low BMD, an individual who is A/A homozygous for the polymorphism is classified as being at the highest risk; an individual who is A/G heterozygous is classified as having moderate risk; an individual who is G/G homozygous is in the lowest risk category.

Use of Functional Polymorphisms

Most preferred for use in the present invention are SNPs which are directly responsible for the BMD phenotype (“functional polymorphisms”). Intronic SNPs may, for example, be situated in regions involved in gene transcripton. SNPs may be directly responsible for the BMD phenotype because of an effect on the amino acid coding, or by disruption of regulatory elements, e.g., which may regulate gene expression, or by disruption of sequences (which may be exonic or intronic) involved in regulation of splicing, such as exonic or splicing enhancers as discussed below.

It is notable that of the two promoter polymorphisms, one (G9326A) is situated at a consensus recognition sequence for the transcription factor AP1 (http://transfac.gbf.de/). In the presence of the G-nucleotide, the consensus AP1 site is present (TCACGGC) on the reverse strand whereas in the presence of the A nucleotide, the consensus sequence is disrupted (TCATGGC).

The A14286G polymorphisms is in intron 4 of the TCIRG1 gene. Two transcripts are derived from the TCIRG1 locus however. The osteoclast specific form (termed ATP6i) is assembled from 20 exons, whereas another transcript termed TIRC7, which is more widely expressed, comprises 14 exons and starts in exon 5 of the osteoclast-specific isoform. Since the A14286G polymorphism is in the proximal promoter of the shorter TCIR7 transcript (intron 4 is only 82 bp long), it may influence transcription or splicing of TCIRG1.

A coding polymorphism in TCIRG1 has been described (at position 2827 on AF033033) which causes an arginine to tryptophan amino acid change at codon 56 (R56W). While this polymorphism was observed in our population, it was rare (allele frequency 0.02) and therefore unlikely to explain the effect observed.

Irrespective of these points and the precise underlying cause of the associations described herein, those skilled in the art will appreciate that the disclosure has great utility for genotyping of BMD in individuals, whether through functional polymorphisms, or polymorphisms which are in linkage disequilibrium with functional polymorphisms (which may be elsewhere in the TCRIG1 locus or in other genes nearby). The invention thus extends to the use not only of the markers described above, but also (for example) to polymorphic markers which are in linkage disequilibrium with any of the markers discussed above, e.g., in linkage disequilibrium with the preferred marker at position 9326.

Use of Other Polymorphisms

As is understood by the person skilled in the art, linkage disequilibrium is the non-random association of alleles. Further details may be found in Kruglyak (1999) Nature Genetics, Vol 22, page 139 and Boehnke (2001) Nature Genetics 25: 246-247). For example, results of recent studies indicate (summarised by Boehnke) that significant linkage disequilibrium may extend for between 0.1 to 0.2 centimorgans.

The five markers described above showed strong and highly significant linkage disequilibrium with each other in our population, with the exception of C14242T where linkage disequilibrium was only observed with A14286G.

Other polymorphic markers which are in linkage disequilibrium with any of the polymorphic markers described above may be identified in the light of the disclosure herein without undue burden by further analysis e.g., within the TCIRG1 gene.

Thus in a related aspect, the present invention provides a method for mapping further polymorphisms which are associated, or are in linkage disequilibrium with a TCIRG1 polymorphism, as described herein. Such a method may preferably be used to identify further polymorphisms associated with variation in BMD. Such a method may involve sequencing of the TCIRG1 gene, or may involve sequencing regions upstream and downstream of the TCIRG1 gene for associated polymorphisms.

In a further aspect, the present invention provides a method of identifying open reading frames which influence BMD. Such a method may comprise screening a genomic sample with an oligonucleotide sequence derived from a TCIRG1 polymorphic marker as described herein and identifying open reading frames proximal to that genetic sequence.

A region which is described as ‘proximal’ to a polymorphic marker may be within about 1000 kb of the marker, preferably within about 500 kb away, and more preferably within about 100 kb, more preferably within 50 kb, more preferably within 10 kb of the marker.

Materials

The invention further provides oligonucleotides for use in probing or amplification reactions, which may be fragments of the sequence shown in Annex I, or a polymorphic variant thereof (see Tables herein).

Preferred primers are as follows:
for the promoter polymorphisms (G9326A and G9508A): Forward: 5′ ACAAGGCAGGCGCAGGACTCC and Reverse: CGGGCCTGGAAACTGAGTCAC;
for the exon 4 (C14242T) and intron 4 (A14286G) polymorphisms: Forward 5′ TTGGGGCAGCAGGTGGGGCC 3′ and Reverse: AGAGGAGAACCCCCTAGGGCTAG 3′;
for the intron 11 polymorphism (G19031A): Forward: GTTCGGGGATGTGGGCCAC 3′/and Reverse: 5′ GCCCATAAGCAGGAGCAGG 3′.

Nucleic acid for use in the methods of the present invention, such as an oligonucleotide probe and/or pair of amplification primers, may be provided in isolated form and may be part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc. The nucleic acid may be labelled. A kit for use in determining the presence or absence of nucleic acid of interest may include one or more articles and/or reagents for performance of the method, such as means for providing the test sample itself, e.g. a swab for removing cells from the buccal cavity or a syringe for removing a blood sample (such components generally being sterile).

The various embodiments of the invention described above may also apply to the following: a diagnostic means for determing the risk of a BMD-related disorder (e.g. osteoporosis); a diagnostic kit comprising such a diagnostic means; a method of osteoporosis therapy, which may include the step of screening an individual for a genetic predisposition to osteoporosis, wherein the predisposition is correlated with a TCIRG1 polymorphic marker, and if a predisposition is identified, treating that individual to prevent or reduce the onset of osteoporosis (such a method may comprise the treatment of the individual by hormone replacement therapy); and the use, in the manufacture of means for assessing whether an individual has a predisposition to osteoporosis, of sequences (e.g., PCR primers) to amplify a region of the TCIRG1 gene.

Assessment of SNPs

Methods for assessment of polymorphisms are reviewed by Schafer and Hawkins, (Nature Biotechnology (1998)16, 33-39, and references referred to therein) and include: allele specific oligonucleotide probing, amplification using PCR, denaturing gradient gel electrophoresis, RNase cleavage, chemical cleavage of mismatch, T4 endonuclease VII cleavage, multiphoton detection, cleavase fragment length polymorphism, E.coli mismatch repair enzymes, denaturing high performance liquid chromatography, (MALDI-TOF) mass spectrometry, analysing the melting characteristics for double stranded DNA fragments as described by Akey et al (2001) Biotechniques 30; 358-367. These references, inasmuch as they be used in the performance of the present invention by those skilled in the art, are specifically incorporated herein by reference.

The assessment of the polymorphism may be carried out on a DNA microchip, if appropriate. One example of such a microchip system may involve the synthesis of microarrays of oligonucleotides on a glass support. Fluorescently-labelled PCR products may then be hybridised to the oligonucleotide array and sequence specific hybridisation may be detected by scanning confocal microscopy and analysed automatically (see Marshall & Hodgson (1998) Nature Biotechnology 16: 27-31, for a review).

Some preferred examples of such methods will now be discussed in more detail.

Use of Nucleic Acid Probes

The method of assessment of the polymorphism may comprise determining the binding of an oligonucleotide probe to the nucleic acid sample. The probe may comprise a nucleic acid sequence which binds specifically to a particular allele of a polymorphism and does not bind specifically to other alleles of the polymorphism. Where the nucleic acid is double-stranded DNA, hybridisation will generally be preceded by denaturation to produce single-stranded DNA. A screening procedure, chosen from the many available to those skilled in the art, is used to identify successful hybridisation events and isolated hybridised nucleic acid.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined.

Binding of a probe to target nucleic acid (e.g. DNA) may be measured using any of a variety of techniques at the disposal of those skilled in the art. For instance, probes may be radioactively, fluorescently or enzymatically labelled. Polymorphisms may be detected by contacting the sample with one or more labelled nucleic acid reagents including recombinant DNA molecules, cloned genes or degenerate variants thereof under conditions favorable for the specific annealing of these reagents to their complementary sequences within the relevant gene. Preferably, the lengths of these nucleic acid reagents are at least 15 to 30 nucleotides. After incubation, all non-annealed nucleic acids are removed from the nucleic acid:gene hybrid. The presence of nucleic acids that have hybridized, if any such molecules exist, is then detected. Using such a detection scheme, the nucleic acid from the cell type or tissue of interest can be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Approaches which rely on hybridisation between a probe and test nucleic acid and subsequent detection of a mismatch may be employed. Under appropriate conditions (temperature, pH etc.), an oligonucleotide probe will hybridise with a sequence which is not entirely complementary. The degree of base-pairing between the two molecules will be sufficient for them to anneal despite a mis-match. Various approaches are well known in the art for detecting the presence of a mis-match between two annealing nucleic acid molecules. For instance, RN'ase A cleaves at the site of a mis-match. Cleavage can be detected by electrophoresing test nucleic acid to which the relevant probe or probe has annealed and looking for smaller molecules (i.e. molecules with higher electrophoretic mobility) than the full length probe/test hybrid. Other approaches rely on the use of enzymes such as resolvases or endonucleases.

Thus, an oligonucleotide probe that has the sequence of a region of the normal gene (either sense or anti-sense strand) in which polymorphisms associated with the trait of interest are known to occur may be annealed to test nucleic acid and the presence or absence of a mis-match determined. Detection of the presence of a mis-match may indicate the presence in the test nucleic acid of a mutation associated with the trait. On the other hand, an oligonucleotide probe that has the sequence of a region of the gene including a mutation associated with disease resistance may be annealed to test nucleic acid and the presence or absence of a mis-match determined. The presence of a mis-match may indicate that the nucleic acid in the test sample has the normal sequence, or a different mutant or allele sequence. In either case, a battery of probes to different regions of the gene may be employed.

As discussed above, suitable probes may comprise all or part of the sequence shown in Annex I (or complement thereof), or all or part of a polymorphic form of the sequence shown in Annex I (or complement thereof (e.g., containing one or more of the polymorphisms shown in the Tables).

Those skilled in the art are well able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42° C. in 6×SSC and washing in 6×SSC at a series of increasing temperatures from 42° C. to 65° C. One common formula for calculating the stringency conditions required to achieve hybridization between nucleic acid molecules of a specified sequence homology is (Sambrook et al., 1989): Tₘ=81.5° C.+16.6Log [Na+]+0.41 (% G+C)−0.63 (% formamide)−600/#bp in duplex.

Other suitable conditions and protocols are described in Molecular Cloning: a Laboratory Manual: 2nd edition, Sambrook et al., 1989, Cold Spring Harbor Laboratory Press and Current Protocols in Molecular Biology, Ausubel et al. eds., John Wiley & Sons, 1992.

Amplification-based Methods

The hybridisation of such a probe may be part of a PCR or other amplification procedure. Accordingly, in one embodiment the method of assessing the polymorphism includes the step of amplifying a portion of the TCIRG1 locus, which portion comprises at least one polymorphism.

The assessment of the polymorphism in the amplification product may then be carried out by any suitable method, e.g., as described herein. An example of such a method is a combination of PCR and low stringency hybridisation with a suitable probe. Unless stated otherwise, the methods of assessing the polymorphism described herein may be performed on a genomic DNA sample, or on an amplification product thereof.

Where the method involves PCR, or other amplification procedure, any suitable PCR primers may be used. The person skilled in the art is able to design such primers, examples of which are shown in Table 3.

An oligonucleotide for use in nucleic acid amplification may be about 30 or fewer nucleotides in length (e.g. 18, 21 or 24). Generally specific primers are upwards of 14 nucleotides in length, but need not be than 18-20. Those skilled in the art are well versed in the design of primers for use processes such as PCR.

Various techniques for synthesizing oligonucleotide primers are ell known in the art, including phosphotriester and phosphodiester synthesis methods.

Suitable polymerase chain reaction (PCR) methods are reviewed, for instance, in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)). PCR comprises steps of denaturation of template nucleic acid (if double-stranded), annealing of primer to target, and polymerisation.

An amplification method may be a method other than PCR. Such methods include strand displacement activation, the QB replicase system, the repair chain reaction, the ligase chain reaction, rolling circle amplification and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

Sequencing

The polymorphism may be assessed or confirmed by nucleotide sequencing of a nucleic acid sample to determine the identity of a polymorphic allele. The identity may be determined by comparison of the nucleotide sequence obtained with a sequence shown in the Annex, Figures and Tables herein. In this way, the allele of the polymorphism in the test sample may be compared with the alleles which are shown to be associated with susceptibility for osteoporosis.

Nucleotide sequence analysis may be performed on a genomic DNA sample, or amplified part thereof, or RNA sample as appropriate, using methods which are standard in the art.

Where an amplified part of the genomic DNA sample is used, the genomic DNA sample may be subjected to a PCR amplification reaction using a pair of suitable primers. In this way the region containing a particular polymorphism or polymorphisms may be selectively amplified (PCR methods and primers are discussed in more detail above). The nucleotide sequence of the amplification product may then be determined by standard techniques.

Other techniques which may be used are single base extension techniques and pyrosequencing.

Mobility Based Methods

The assessment of the polymorphism may be performed by single strand conformation polymorphism analysis (SSCP). In this technique, PCR products from the region to be tested are heat denatured and rapidly cooled to avoid the reassociation of complementary strands. The single strands then form sequence dependent conformations that influence gel mobility. The different mobilities can then be analysed by gel electrophoresis.

Assessment may be by heteroduplex analysis. In this analysis, the DNA sequence to be tested is amplified, denatured and renatured to itself or to known wild-type DNA. Heteroduplexes between different alleles contain DNA “bubbles” at mismatched basepairs that can affect mobility through a gel. Therefore, the mobility on a gel indicates the presence of sequence alterations.

Restriction Site Based Methods

Where an SNP creates or abolishes a restriction site, the assessment may be made using RFLP analysis. In this analysis, the DNA is mixed with the relevant restriction enzyme (i.e., the enzyme whose restriction site is created or abolished). The resultant DNA is resolved by gel electrophoresis to distinguish between DNA samples having the restriction site, which will be cut at that site, and DNA without that restriction site, which will not be cut.

Where the SNP does not create or abolish a restriction site the SNP may be assessed in the following way. A mutant PCR primer may be designed which introduces a mutation into the amplification product, such that a restriction site is created when one of the polymorphic variants is present but not when another polymorphic variant is present. After PCR amplification using this primer (and another suitable primer), the amplification product is admixed with the relevant restriction enzyme and the resultant DNA analysed by gel electrophoresis to test for digestion.

The invention will now be further described with reference to the following non-limiting Figure, Example, Tables and Annex. Other embodiments of the invention will occur to those skilled in the art in the light of these.

### FIGURES

FIG. 1 shows the TCIRG1 gene structure and location of polymorphisms. Common haplotypes with allele frequency greater than 5% are shown. The translation start site (CDS) of the osteoclast specific isoform is indicated.

### EXAMPLES OF BMD-RELATED TCIRG1 POLYMORPHISMS

Subjects

The study group comprised 739 unrelated women aged 45-55 who were randomly selected from a large population based BMD screening programme for osteoporotic fracture risk [15]. This screening program originally involved 7000 women who were identified using Community Health Index records (CHI) from a 25-mile radius of Aberdeen, a city with a population of about 250,000 in the North East of Scotland. Women were invited by letter to undergo BMD measurements between 1990-1994 and 5119 of the 7000 invited (73.1%) attended for evaluation. Blood samples were subsequently obtained for DNA extraction on 3069 (59.9%) of these individuals. Participants were weighed wearing light clothing and no shoes on a set of balance scales calibrated to 0.05 kg (Seca, Hamburg, Germany). Height was measured using a stadiometer (Holtain Ltd, Crymych, United Kingdom). Participants completed a questionnaire on menopausal status, and use of Hormone Replacement Therapy (HRT) and on the basis of this, were classified into five groups. Women were classified as “premenopausal” if they were not on HRT and menstruating regularly (n=374), as “perimenopausal” if they were not on HRT and menstruation was irregular and/or if up to 6 months had elapsed since their last period (n=14) and “postmenopausal” if they were not on HRT and menstruation had ceased for 6 months or more (n=144). The remaining two groups consisted of women who were currently receiving HRT at the time of study (n=196) and those who previously had received HRT (n=11). Current and previous HRT users were not further classified in terms of menopausal status.

All participants gave written informed consent to being included in the study which was approved by the Grampian Joint Research Ethical Committee.

Bone Mineral Densitometry

Bone mineral density measurements (BMD) of the left proximal femur (the femoral neck, FN) and lumbar spine, LS (L2-4) were performed by dual energy x-ray absorptiometry using one of two Norland XR26 or XR36 densitometers (Norland Corp, Wisconsin, USA). Calibration of the machines was performed daily, and quality assurance checked by measuring the manufacturer's lumbar spine phantom at daily intervals and a Hologic spine phantom at weekly intervals. The in-vivo precision for the XR36 was 1.2% for the lumbar spine (LS), and 2.3% for the femoral neck (FN). Corresponding values for the XR26 were 1.95% and 2.31% (LS and FN respectively).

Mutation Screening and Genotyping

Mutation screening was carried out by DNA sequencing of the promoter and intron-exon boundaries of the TCIRG1 gene in DNA extracted from peripheral venous blood samples from about 70 individuals using PCR based methods as previously described [13;14]. Genotyping for polymorphisms was carried out by DNA sequencing of PCR amplified fragments of genomic DNA. The PCR products for sequencing were generated using Qiagen Taq DNA polymerase, Q-solution and standard reaction buffer containing 1.5mM MgCl₂ according to the manufacturer's recommendations. The PCR was carried out for 35 cycles with a melting temperature of 95° C., an annealing temperature of 60° C. and an extension temperature of 72° C.

The promoter polymorphisms (G9326A and G9508A); were analysed using the following primer pairs: Forward: 5′ ACAAGGCAGGCGCAGGACTCC and Reverse: CGGGCCTGGAAACTGAGTCAC; the exon 4 (C14242T) and intron 4 (A14286G) polymorphisms were analysed using the following primer pairs: Forward 5′ TTGGGGCAGCAGGTGGGGCC 3′ and Reverse: AGAGGAGAACCCCCTAGGGCTAG 3′; and the intron 11 polymorphism (G19031A) was analysed using the following primer pairs: Forward: GTTCGGGGATGTGGGCCAC 3′/and Reverse: 5′ GCCCATAAGCAGGAGCAGG 3′. The PCR products were treated with Exonuclease III and Shrimp Alkaline Phosphatase-(Exo-SAP-IT) (Amersham Pharmacia) according to the manufacturers instructions and sequenced using the forward and/or reverse primer as the sequencing primer using DYNamic ET sequencing chemistry on a MegaBace 1000 DNA sequencer (Amersham Pharmacia)

Statistical Methods

Statistical analysis was carried out using Minitab version 12 (Minitab Inc, Pennsylvania, USA). Differences in BMD between the genotypes were tested using one way ANOVA and General Linear Model (GLM) analysis of variance (ANOVA) adjusting for height, weight, age, menopausal status/HRT use and smoking. Haplotypes were constructed from the population genotype data by the algorithm of Niu and colleagues, using the Haplotyper program [Liu et al, (2002) Am.J Hum.Genet. 70:157-169]. GLM ANOVA analysis was also used to test for allelic associations, by combining data from the genotype groups and for haplotypes predicted by the Haplotyper program. Stepwise logistic regression was used to evaluate the relative contribution of genotype and other factors to the population variance in BMD. Linkage disequilibrium between polymorphisms was estimated by calculating D′ values using the 2BY2 program on output generated by the EH program [Terwilliger J D, Ott J (1994) Handbook of Human Genetic Linkage. Johns Hopkins University Press, Baltimore & London]. Both programs were obtained from the Columbia University Website.

Results

We identified 5 common polymorphisms (those with allele frequency greater than 5%) in TCIRG1 on mutation screening of 70 normal subjects. These were: a C to T change at position 14242, (C14242T) an A to G change at position 14286 (Al4286G) and a G to A change at position 19031 (G19031A) [which are positions 3856, 3900 and 8645 on sequence accession number AF033033].

The C14242T change is within exon 4 of the osteoclast specific transcript of the TCIRG1 gene but is a conservative change (CAC-CAT; both histidine). The A14286G polymorphisms is within intron 4 of TCIRG1 and the G19031A is within intron 11 (G19031A). Two additional polymorphisms were discovered in the TCIRG1 promoter. These are at positions 9326 (G9326A) and 9508 (G9508A) on sequence accession number AP002807 in which the first nucleotide of the TCIRG1 mRNA start site is assumed to be position 10428. We did not detect any of the exonic polymorphisms present in the SNP database cited by Carn et al[supra] with the exception of the C226T change which predicts an arginine to tryptophan amino change at codon 56 (this is at position 2827 of sequence accession number AF033033). This was rare however, with an allele frequency of only 2% in the normal subjects used for mutation screening and was not analysed further in the population based study.

Details of age, BMD, height, weight, smoking history, menopausal status, and HRT use in the study population are shown in Table 1. 50.6% of the women were premenopausal, 1.8% perimenopausal and 19.4% were postmenopausal. The average time elapsed since menopause was 6.07 years in the postmenopausal group. Menopausal status was unclassified for 207 (28%) of subjects because the date of cessation of natural menstruation could not be accurately established because of current HRT use in 196 women (26.5%) and previous HRT use in 11 women (1.4%).

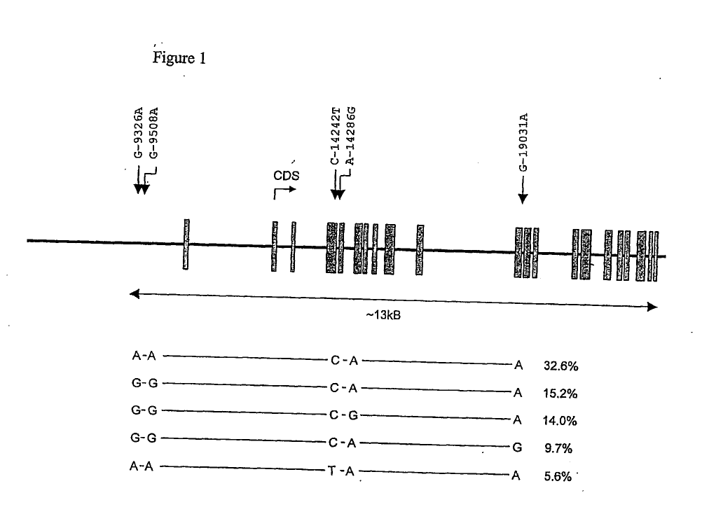

Significant linkage disequilibrium (LD) was observed between most of the polymorphisms identified. The strongest LD was between G9326A and G9508A (D′=0.80, p<0.0001). Other LD values ranged between 0.569-0.752 (all p<0.001), with the exception of the C14242T polymorphism which showed significant LD only with A14286G. (D′=0.321; p<0.001). Analysis using the Haplotyper program predicted 27 different haplotypes from the genotype data, but five common haplotypes were identified that accounted for 77.3% of alleles at the TCIRG1 locus. These are summarised in FIG. 1, which also illustrates the position of the polymorphisms in relation to the TCIRG1 gene structure.

We studied the relationship between genotypes at each site and BMD values, before and after adjustment for age, height, weight, menopausal status/HRT use and smoking. The results of this analysis are shown in Table 2 for spine BMD and Table 3 for hip BMD. The genotype distributions of G9326A, G9508A, A14286G and G19031A were as predicted by Hardy-Weinberg equilibrium, but for the C14242T polymorphism, we found more C/T heterozygotes than expected (102 vs 64, p=0.007).

There was a significant association between G9326A polymorphism and both spine and hip BMD. The differences were significant for unadjusted and adjusted BMD values. When data were combining for the G/A heterozygotes and A/A homozygotes, the difference between groups was also significant at the spine and hip for adjusted BMD.

A non-significant trend for association between the C14242T polymorphism and adjusted spine BMD values was observed (p=0.079) and this became significant when the C/T and T/T genotypes were combined (p=0.036). None of the other polymorphisms was associated with BMD, nor did we find a significant association between any of the TCIRG1 haplotypes predicted by the Haplotyper program and BMD (data not shown). There was no association between TCIRG1 genotype and age, weight, height, smoking or menopausal status (data not shown).

We also studied the relationship between TCIRG1 genotypes in relation to menopausal status and HRT use. This analysis was restricted to premenopausal women, postmenopausal women and current HRT users in view of the small number of subjects in the perimenopausal and previous HRT user groups. There was no significant association between G9508A, C14242T, A14286G or G19031A genotypes and BMD in any of these subgroups, nor was there an association between TCIRG1 haplotypes and BMD (data not shown). The G9326A polymorphism was significantly associated with BMD in the subgroup of women who were pre-menopausal, but there was no association between G9326A and BMD in postmenopausal women or HRT users (Table 4).

Analysis of the data by stepwise multiple regression identified three independent predictors of spine BMD, which together accounted for 13.3% of the variance in spine BMD. These were body weight (9.41% of the variance, p<0.0001); menopausal status/HRT use (3.16% of the variance, p<0.0001); and the G9326A allele (1.00% of the variance, p=0.017). For femoral neck BMD, we identified two independent predictors which accounted for 11.1% of the variance. These were body weight (13.6% of the variance, p<0.0001) and menopausal status/HRT use (0.85% of the variance, p=0.009).

TABLE 1
Demographic details of study population

| Table 1 | | |
|---|---|---|
| Number | 739 | |
| Age | 47.9 ± 1.53 | |
| Premenopausal | 374 | (50.6%) |
| Perimenopausal | 14 | (1.9%) |
| Postmenopausal | 144 | (19.5%) |
| Previous HRT users | 11 | (1.5%) |
| Current HRT users | 196 | (26.5%) |

| | |
|---|---|
| Years since menopause* | 6.1 ± 5.3 |
| Spine BMD (g/cm²) | 1.067 ± 0.15 |
| Femoral Neck BMD | 0.890 ± 0.12 |
| (g/cm²) | |
| Weight | 65.9 ± 11.8 |
| Height | 161.5 ± 6.1 |

Values are means and SD or numbers and percentages.
*in postmenopausal women

TABLE 2
Lumbar spine BMD values in relation to TCIRG1 genotypes and alleles

| | | | | | ANOVA | ANOVA |
|---|---|---|---|---|---|---|
| | Genotypes | | | Allele | (genotypes) | (alleles) |
| G9326A | GG (n = 136) | GA (n = 309) | AA (n = 163) | GA/AA | p-value | p-value |
| | | | | (n = 472) | | |
| (unadjusted BMD) | 1.099 ± 0.173 | 1.052 ± 0.158 | 1.080 ± 0.140 | 1.062 ± 0.152 | 0.011 | 0.017 |
| (adjusted BMD) | 1.074 ± 0.018 | 1.027 ± 0.016 | 1.049 ± 0.018 | 1.035 ± 0.015 | 0.008 | 0.007 |
| G9508A | GG (n = 121) | GA (n = 333) | AA (n = 193) | GA/AA (n = 526) | | |
| (unadjusted BMD) | 1.079 ± 0.181 | 1.059 ± 0.156 | 1.077 ± 0.146 | 1.065 ± 0.153 | 0.311 | 0.379 |
| (adjusted BMD) | 1.061 ± 0.017 | 1.039 ± 0.014 | 1.052 ± 0.016 | 1.043 ± 0.014 | 0.283 | 0.244 |
| C14242T | CC (n = 495) | CT (n = 102) | TT (n = 2) | CT/TT (n = 104) | | |
| (unadjusted BMD) | 1.072 ± 0.157 | 1.043 ± 0.148 | 1.070 ± 0.092 | 1.044 ± 0.147 | 0.240 | 0.094 |
| (adjusted BMD) | 1.049 ± 0.014 | 1.014 ± 0.019 | 1.101 ± 0.103 | 1.015 ± 0.020 | 0.079 | 0.036 |
| A14286G | AA (n = 346) | AG (n = 213) | GG (n = 42) | AG/GG (n = 255) | | |
| (unadjusted BMD) | 1.095 ± 0.200 | 1.054 ± 0.151 | 1.074 ± 0.150 | 1.074 ± 0.150 | 0.187 | 0.326 |
| (adjusted BMD) | 1.054 ± 0.015 | 1.031 ± 0.016 | 1.072 ± 0.025 | 1.039 ± 0.015 | 0.109 | 0.200 |
| G19031A | GG (n = 428) | GA (n = 160) | AA (n = 21) | GA/AA (n = 181) | | |
| (unadjusted BMD) | 1.064 ± 0.153 | 1.066 ± 0.167 | 1.128 ± 0.161 | 1.073 ± 0.167 | 0.189 | 0.530 |
| (adjusted BMD) | 1.038 ± 0.014 | 1.042 ± 0.016 | 1.102 ± 0.034 | 1.048 ± 0.016 | 0.170 | 0.394 |

Unadjusted BMD values are mean±SD in g/cm². Adjusted BMD values are least squares mean±SD BMD values adjusted for age, weight, height, menopausal status/HRT use and smoking.

TABLE 3
Femoral neck BMD values in relation to TCIRG1 genotypes and alleles

| | | | | | ANOVA | ANOVA |
|---|---|---|---|---|---|---|
| | Genotypes | | | Alleles | (genotypes) | (alleles) |
| G9326A | GG (n = 136) | GA (n = 309) | AA (n = 164) | GA/AA (n = 473) | p-value | p-value |
| (unadjusted BMD) | 0.907 ± 0.144 | 0.879 ± 0.119 | 0.904 ± 0.123 | 0.888 ± 0.121 | 0.042 | 0.126 |
| (adjusted BMD) | 0.896 ± 0.014 | 0.866 ± 0.013 | 0.886 ± 0.014 | 0.873 ± 0.012 | 0.030 | 0.047 |
| G9508A | GG (n = 121) | GA (n = 333) | AA (n = 194) | GA/AA (n = 527) | | |
| (unadjusted BMD) | 0.902 ± 0.147 | 0.880 ± 0.121 | 0.9508 ± 0.121 | 0.888 ± 0.121 | 0.125 | 0.254 |
| (adjusted BMD) | 0.895 ± 0.014 | 0.871 ± 0.011 | 0.884 ± 0.013 | 0.875 ± 0.011 | 0.114 | 0.104 |
| C14242T | CC (n = 496) | CT (n = 102) | TT (n = 2) | CT/TT (n = 104) | | |
| (unadjusted BMD) | 0.894 ± 0.126 | 0.873 ± 0.117 | 0.865 ± 0.087 | 0.873 ± 0.117 | 0.301 | 0.122 |
| (adjusted BMD) | 0.874 ± 0.011 | 0.848 ± 0.015 | 0.897 ± 0.082 | 0.849 ± 0.015 | 0.130 | 0.053 |
| A14286G | AA (n = 347) | AG (n = 213) | GG (n = 42) | AG/GG (n = 255) | | |
| (unadjusted BMD) | 0.886 ± 0.123 | 0.888 ± 0.122 | 0.909 ± 0.145 | 0.892 ± 0.126 | 0.555 | 0.611 |
| (adjusted BMD) | 0.891 ± 0.011 | 0.905 ± 0.013 | 0.932 ± 0.026 | 0.872 ± 0.012 | 0.474 | 0.787 |
| G19031A | GG (n = 429) | GA (n = 160) | AA (n = 21) | GA/AA (n = 181) | | |
| (unadjusted BMD) | 0.895 ± 0.121 | 0.876 ± 0.132 | 0.914 ± 0.130 | 0.881 ± 0.132 | 0.195 | 0.211 |
| (adjusted BMD) | 0.878 ± 0.011 | 0.862 ± 0.012 | 0.9508 ± 0.027 | 0.865 ± 0.012 | 0.179 | 0.234 |

Unadjusted BMD values are mean±SD in g/cm². Adjusted BMD values are least squares mean±SD BMD values adjusted for age, weight, height, menopausal status/HRT use and smoking.

TABLE 4
TCIRG1 G9326A alleles and BMD in relation to HRT use and menopausal status.

| | G9326A | | | G9326A | ANOVA | ANOVA |
|---|---|---|---|---|---|---|
| | Genotypes | | | Alleles | (genotypes) | (alleles) |
| Spine BMD | GG | GA | AA | GA/AA | p-value | p-value |
| Premenopausal (n = 308) | (n = 67) | (n = 157) | (n = 83) | (n = 240) | | |
| adjusted BMD | 1.142 ± 0.020 | 1.075 ± 0.013 | 1.099 ± 0.019 | 1.082 ± 0.012 | 0.011 | 0.006 |
| Postmenopausal (n = 119) | (n = 29) | (n = 52) | (n = 37) | (n = 89) | | |
| adjusted BMD | 1.085 ± 0.026 | 1.065 ± 0.021 | 1.028 ± 0.024 | 1.049 ± 0.016 | 0.230 | 0.228 |
| HRT users (n = 164) | (n = 32) | (n = 94) | (n = 37) | (n = 141) | | |
| adjusted BMD | 1.025 ± 0.025 | 1.016 ± 0.014 | 1.064 ± 0.024 | 1.028 ± 0.013 | 0.215 | 0.863 |
| Femoral Neck BMD | GG | GA | AA | GA/AA | | |
| Premenopausal (n = 308) | (n = 67) | (n = 157) | (n = 84) | (n = 240) | | |
| adjusted BMD | 0.934 ± 0.015 | 0.882 ± 0.010 | 0.903 ± 0.014 | 0.888 ± 0.009 | 0.013 | 0.008 |
| Postmenopausal (n = 119) | (n = 29) | (n = 52) | (n = 37) | (n = 89) | | |
| adjusted BMD | 0.896 ± 0.020 | 0.904 ± 0.016 | 0.888 ± 0.018 | 0.897 ± 0.013 | 0.789 | 0.947 |
| HRT users (n = 164) | (n = 32) | (n = 94) | (n = 37) | (n = 141) | | |
| adjusted BMD | 0.869 ± 0.021 | 0.869 ± 0.012 | 0.9508 ± 0.020 | 0.877 ± 0.011 | 0.379 | 0.708 |

Adjusted BMD values are least squares mean±SD BMD values adjusted for age, weight, height, menopausal status/HRT use and smoking

### REFERENCES

1. Kanis, J. A., Melton, L. J., Christiansen, C., Johnston, C. C., and Khaltaev, N. (1994) The diagnosis of osteoporosis. J Bone Miner Res, 9,1137-1141.

2. Gueguen, R., Jouanny, P., Guillemin, F., Kuntz, C., Pourel, J., and Siest, G. (1995) Segregation analysis and variance components analysis of bone mineral density in healthy families. J Bone Miner Res, 12,2017-2022.

3. Arden, N. K., Spector, T. D. (1997) Genetic influences on muscle strength, lean body mass, and bone mineral density: a twin study. J Bone Miner Res, 12,2076-2081.

4. Smith, D. M., Nance, W. E., Kang, K. W., Christian, J. C., and Johnston, C. C. (1973) Genetic factors in determining bone mass. J Clin Invest, 52,2800-2808.

5. Morrison, N. A., Qi, J. C., Tokita, A., Kelly, P., Crofts, L., Nguyen, T. V., Sambrook, P. N., and Eisman, J. A. (1997) Prediction of bone density from vitamin D receptor alleles (Erratum). Nature, 387,106-106.

6. Kobayashi, S., Inoue, S., Hosoi, T., Ouchi, Y., Shiraki, M., and Orimo, H. (1996) Association of bone mineral density with polymorphism of the estrogen receptor gene. J.Bone Miner.Res., 11,306-311.

7. Grant, S. F. A., Reid, D. M., Blake, G., Herd, R., Fogelman, I., and Ralston, S. H. (1996) Reduced bone density and osteoporosis associated with a polymorphic Spl site in the collagen type I alpha 1 gene. Nature Genetics, 14,203-205.

8. Rubin, L. A., Hawker, G. A., Peltekova, V. D., Fielding, L. J., Ridout, R., and Cole, D. E. (1999) Determinants of peak bone mass: clinical and genetic analyses in a young female Canadian cohort. Journal of Bone & Mineral Research, 14,633-643.

9. Gong, Y., Vikkula, M., Boon, L., Lui, J., Beighton, P., Ramesar, R., Peltonen, L., Somer, H., Hirose, T., Dallapicola, B., De Paepe, A., Swoboda, W., Zabel, B., Superti-Furga, A., Steinmann, B., Brunner, H. G., Jans, A., Boles, R. G., Adkins, W., van den Boogard, M. J., Olsen, B. R., and Warman, M. L. (1998) osteoporosis-pseudoglioma syndrome, a disorder affecting skeletal strength and vision, is assigned to chromosome region 11q12-13. Am J Hum Genet, 1996,146-151.

10. Heaney, C., Shalev, H., Elbedour, K., Carmi, R., Staack, J. B., Sheffield, V. C., and Beier, D. R. (1998) Human autosomal recessive osteopetrosis maps to 11q13, a position predicted by comparative mapping of the murine osteosclerosis (oc) mutation. Human Molecular Genetics, 7,1407-1410.

11. Johnson, M. L., Gong, G., Kimberling, W., Recker, S., Kimmel, D. B., and Recker, R. R. (1997) Linkage of a gene causing high bone mass to human chromosome 11 (11q12-13). Am J Hum Genet, 60,1326-1332.

12. Koller, D. L., Econs, M. J., Morin, P. A., Christian, J. C., Hui, S. L., Parry, P., Curran, M. E., Rodriguez, L. A., Conneally, P. M., Joslyn, G., Peacock, M., Johnston, C. C., and Foroud, T. (2000) Genome Screen for QTLs Contributing to Normal Variation in Bone Mineral Density and Osteoporosis. J Clin Endocrinol Metab, 85,3116-3120.

13. Frattini, A., Orchard, P. J., Sobacchi, C., Giliani, S., Abinun, M., Mattsson, J. P., Keeling, D. J., Andersson, A. K., Wallbrandt, P., Zecca, L., Notarangelo, L. D., Vezzoni, P., and Villa, A. (2000) Defects in TCIRG1 subunit of the vacuolar proton pump are responsible for a subset of human autosomal recessive osteopetrosis. Nat.Genet., 25,343-346.

14. Sobacchi, C., Frattini, A., Orchard, P., Porras, O., Tezcan, I., Andolina, M., Babul-Hirji, R., Baric, I., Canham, N., Chitayat, D., Dupuis-Girod, S., Ellis, I., Etzioni, A., Fasth, A., Fisher, A., Gerritsen, B., Gulino, V., Horwitz, E., Klamroth, V., Lanino, E., Mirolo, M., Musio, A., Matthijs, G., Nonomaya, S., Notarangelo, L. D., Ochs, H. D., Superti, F. A., Valiaho, J., van Hove, J. L., Vihinen, M., Vujic, D., Vezzoni, P., and Villa, A. (2001) The mutational spectrum of human malignant autosomal recessive osteopetrosis. Hum.Mol.Genet., 10,1767-1773.

15. Garton, M J., Torgerson, D J., Donaldson, C., Russell, I T., and Reid, D. M. (1992) Recruitment techniques for screening programmes: trial of a new method within a regional osteoporosis study. Br Med J, 305,82-84.

16. Garton, M. J., Torgerson, D. J., Donaldson, C., Russell, I. T., and Reid, D. M. (1992) Recruitment methods for screening programmes: trial of a new method within a regional osteoporosis study [see comments]. Br Med J, 305,82-84.

17. Vaananen, H. K., Zhao, H., Mulari, M., and Halleen, J. M. (2000) The cell biology of osteoclast function. J. Cell Sci., 113,377-381.

18. Li, Y. P., Chen, W., Liang, Y., Li, E., and Stashenko, P. (1999) Atp6i-deficient mice exhibit severe osteopetrosis due to loss of osteoclast-mediated extracellular acidification. Nat.Genet., 23,447-451.

19. Scimeca, J. C., Franchi, A., Trojani, C., Parrinello, H., Grosgeorge, J., Robert, C., Jaillon, O., Poirier, C., Gaudray, P., and Carle, G. F. (2000) The gene encoding the mouse homologue of the human osteoclast-specific 116-kDa V-ATPase subunit bears a deletion in osteosclerotic (oc/oc) mutants. Bone, 26,207-213.

20. Koller, D. L., Rodriguez, L. A., Christian, J. C., Slemenda, C. W., Econs, M. J., Hui, S. L., Morin, P., Conneally, P. M., Joslyn, G., Curran, M. E., Peacock, M., Johnston, C. C., and Foroud, T. (1999) Linkage of a QTL contributing to normal variation in bone mineral density to chromosome 11q12-13. J Bone Miner Res, 13,1903-1908.

21. Stewart, T. L., Ralston, S. H. (2000) Role of genetic factors in the pathogenesis of osteoporosis. J.Endocrinol., 166,235-245.

22. Willing, M., Sowers, M., Aron, D., Clark, M. K., Burns, T., Bunten, C., Crutchfield, M., D'Agostino, D., and Jannausch, M. (1998) Bone mineral density and its change in white women: estrogen and vitamin D receptor genotypes and their interaction. J. Bone Miner.Res., 13,695-705.

23. Cooper, G. S., Umbach, D. M. (1996) Are vitamin D receptor polymorphisms associated with bone mineral density? A meta-analysis. J Bone Miner Res, 11,1841-1849.

24. Mann, V., Hobson, E. E., Li, B., Stewart, T. L., Grant, S. F., Robins, S. P., Aspden, R. M., and Ralston, S. H. (2001) A COL1A1 Spl binding site polymorphism predisposes to osteoporotic fracture by affecting bone density and quality. J. Clin.Invest, 107,899-907.

1. A method for assessing bone mass in an individual, the method comprising use of a T-cell immune regulator 1 (TCIRG1) marker.

2. A method as claimed in claim 1 for assessing bone mineral density (BMD).

3. A method as claimed in claim 2 wherein the method comprises
(i)obtaining a sample of nucleic acid from an individual, and
(ii) assessing a polymorphic marker in the TCIRG1 sequence of the nucleic acid, plus optionally one or more further steps to attribute a likely BMD value to the individual.

4. A method as claimed in claim 1 wherein the marker is a polymorphic marker.

5. A method as claimed in claim 3 wherein the nucleic acid is genomic DNA.

6. A method as claimed in claim 5 wherein the marker is a single nucleotide polymorphism (SNP) selected from the group consisting of the following positions numbered in accordance with the ap002807 accession: (i) 9326; (ii) 9508; (iii) 14242; (iv) 14286; (v) 19031, or a polymorphic marker which is in linkage disequilibrium with any of these.

7. A method as claimed in claim 6 wherein the SNP is at position 9326, or is an SNP in linkage disequilibrium with said SNP9326.

8. A method as claimed in claim 7 identity of the nucleotide at the SNP is assessed.

9. A method as claimed in claim 8 wherein the identity of the nucleotide at the SNP is one of the following alleles: (i) G9326A; (ii) G9508A; (iii) C14242T; (iv) A14286G; (v) G19031A.

10. A method as claimed in claim 1 wherein the individual is considered at risk from BMD-associated disorder.

11. A method for determining the susceptibility of an individual to a disorder which is related to low BMD, the method comprising use of a method as claimed in claim 1.

12. A method as claimed in claim 11 wherein the disorder is associated with a low lower lumbar spine or low femoral neck BMD.

13. A method as claimed in claim 12 wherein the disorder is osteoporosis.

14. A method as claimed in claim 13 wherein non TCIRG1 polymorphic markers which are linked or associated with low BMD are assessed.

15. A method for assessing the risk of osteoporotic fracture in an individual, the method comprising use of a method as claimed in claim 9.

16. A method as claimed in claim 15 wherein
an individual who is A/A homozygous for SNP9326 or who is A/G heterozygous is classified as having increased risk and
an individual who is G/G homozygous is classified as having lowest risk,
of susceptibility to a disorder which is associated with an abnormally low BMD.

17. A method as claimed in claim 4 wherein the TCIRG1 sequence in assessed by determining the binding of an oligonucleotide probe to the nucleic acid sample, wherein the probe comprises all or part of (i) the TCIRG1 genomic sequence of Annex I, or (ii) a polymorphic form of the TCIRG1 genomic sequence shown in Annex I, or (iii) the complement of either.

18. A method as claimed in claim 17 wherein the probe comprise a nucleic acid sequence which binds under stringent conditions specifically to one particular allele of the TCIRG1 polymorphic marker and does not bind specifically to another alleles of the TCIRG1 polymorphic marker.

19. A method as claimed in claim 18 wherein the probe is labelled and binding of the probe is determined by presence of the label.

20. A method as claimed in claim 4 wherein the method comprises amplifying a region of the TCIRG1 sequence comprising at least one polymorphic marker.

21. A method as claimed in claim 20 wherein a region of the TCIRG1 sequence is amplified by use of two oligonucleotide primers.

22. A method as claimed in claim 21 wherein at least one of said primers binds under stringent conditions specifically to one particular allele of the TCIRG1 polymorphic marker and does not bind specifically to another alleles of the TCIRG1 polymorphic marker.

23. A method as claimed in claim 21 wherein at least one of said primers is a mutagenic primer which introduces a restriction site into said amplified region of the TCIRG1 sequence.

24. A method as claimed in claim 21 wherein at least one of said primers is selected from:

| | | |
|---|---|---|
| Forward: | ACAAGGCAGGCGCAGGACTCC; | (SEQ ID NO:2) |
| Reverse: | CGGGCCTGGAAACTGAGTCAC; | (SEQ ID NO:3) |
| Forward: | TTGGGGCAGCAGGTGGGGCC; | (SEQ ID NO:4) |
| Reverse: | AGAGGAGAACCCCCTAGGGCTAG; | (SEQ ID NO:5) |
| Forward: | GTTCGGGGATGTGGGCCAC; | (SEQ ID NO:6) |
| Reverse: | GCCCATAAGCAGGAGCAGG. | (SEQ ID NO:7) |

25. A method as claimed in claim 4 wherein the TCIRG1 sequence in assessed by a method selected from the group consisting of: strand conformation polymorphic marker analysis; heteroduplex analysis; RFLP analysis.

26. A method as claimed in claim 4 wherein the polymorphic marker is assessed or confirmed by nucleotide sequencing,

27. A method of determining the presence or absence in a test sample of a polymorphic marker in the TCIRG1 sequence which is a single nucleotide polymorphism (SNP) selected from the group consisting of the following positions numbered in accordance with the ap002807 accession: (i) 9326; (ii) 9508; (iii) 14242; (iv) 14286; (v) 19031, which method comprises determining the binding of an oligonucleotide probe to the nucleic acid sample, wherein the probe comprises all or part of (i) the TCIRG1 genomic sequence of Annex I, or; (ii) a polymorphic form of the TCIRG1 genomic sequence shown in Annex I, or; (iii) the complement of either.

28. A method of determining the presence or absence in a test sample of a polymorphic marker in the TCIRG1 sequence which is a single nucleotide polymorphism (SNP) selected from the group consisting of the following positions numbered in accordance with the ap002807 accession: (i) 9326; (ii) 9508; (iii) 14242; (iv) 14286; (v) 19031, which method comprises use of two oligonucleotide primers capable of amplifying a portion of the TCIRG1 sequence which portion comprises at least one of said SNPs.

29. A method for mapping polymorphic markers which are associated with a disorder which is associated with an abnormal level of bone mineral density (BMD), the method comprising identifying polymorphic markers which are in linkage disequilibrium with an SNP which is selected from the group consisting of the following positions numbered in accordance with the ap002807 accession: (i) 9326; (ii) 9508; (iii) 14242; (iv) 14286; (v) 19031,

30. An oligonucleotide probe for use in a method of claim 21.

31. An oligonucleotide probe as claimed in claim 30 which comprises a TCIRG1 polymorphic marker which is a single nucleotide polymorphism (SNP) selected from the group consisting of the following positions numbered in accordance with the ap002807 accession: (i) 9326; (ii) 9508; (iii) 14242; (iv) 14286; (v) 19031,

32. An oligonucleotide probe as claimed in claim 30 which comprises a label.

33. A PCR primer pair for use in a method of claim 21 which primer pair comprises first and second primers which hybridise to DNA in regions including or flanking the TCIRG1 polymorphic marker.

34. A PCR primer pair as claimed in claim 33 wherein the TCIRG1 polymorphic marker is a single nucleotide polymorphism (SNP) selected from the group consisting of the following positions numbered in accordance with the ap002807 accession: (i) 9326; (ii) 9508; (iii) 14242; (iv) 14286; (v) 19031,

35. A PCR primer pair as claimed in claim 34 wherein at least one primer is selected from:

| | | |
|---|---|---|
| Forward: | ACAAGGCAGGCGCAGGACTCC; | (SEQ ID NO:2) |
| Reverse: | CGGGCCTGGAAACTGAGTCAC; | (SEQ ID NO:3) |
| Forward: | TTGGGGCAGCAGGTGGGGCC; | (SEQ ID NO:4) |
| Reverse: | AGAGGAGAACCCCCTAGGGCTAG; | (SEQ ID NO:5) |
| Forward: | GTTCGGGGATGTGGGCCAC; | (SEQ ID NO:6) |
| Reverse: | GCCCATAAGCAGGAGCAGG. | (SEQ ID NO:7) |

36. A kit comprising a probe andor primer of claim 30.

37. A method of osteoporosis prognosis, which method includes the step of screening an individual for a genetic predisposition to osteoporosis in accordance with the method of claim 13, whereby the predisposition is correlated with a TCIRG1 polymorphic marker, and if a predisposition is identified, treating that individual to prevent or reduce the onset of osteoporosis.

38. A method as claimed in claim 37 wherein said treatment comprises hormone replacement therapy.
